(19) United States
(12) Patent Application Publication
(10) Pub. No.: US 2017/0245545 A1
(43) Pub. Date: Aug. 31, 2017

(54) FILTERING PIPE SMOKE

(71) Applicant: Chancellor Proprietary, Inc., New York, NY (US)

(72) Inventors: Darrell Johnson, III, New York, NY (US); Marie Johnson, New York, NY (US)

(21) Appl. No.: 15/056,114

(22) Filed: Feb. 29, 2016

(51) Int. Cl. A24F 1/00; A24F 7/02; F16K 15/14; A24F 5/10; B01D 39/06; B01D 46/30; A61M 15/00; A24F 13/00

(52) U.S. Cl. CPC A24F 1/00; A61M 15/0021; A61M 15/0016; A61M 15/0018; A24F 7/02; A24F 13/00; A24F 5/10; B01D 39/06; B01D 46/30; F16K 15/14; F16K 15/147; A61M 2205/75; B01D 2239/0208

(57) ABSTRACT

A smoking pipe has a a combustion bowl, a mouthpiece, and an inhalation conduit. An inhalation check valve is connected to the inhalation conduit and positioned between the bowl and the mouthpiece, oriented to pass smoke of combustion in one direction from the bowl to the mouthpiece. An exhalation check valve is positioned between the mouthpiece and the atmosphere, and is oriented to pass exhalation in one direction from the mouthpiece to the atmosphere. A body is partially fillable to retain water, and the inhalation conduit passes from the bowl into the body and extends to an upper area inside the body, and then turns to extend towards a lower area inside the body, terminating in an open end. The inhalation conduit has a second portion that has an opening in the upper inner area and extends to the inhalation check valve.

### FIELD OF THE DISCLOSURE

The disclosure relates to a system and method for filtering pipe smoke, and in particular, using a filter for exhaled smoke.

### BACKGROUND OF THE DISCLOSURE

Smoking pipes enable the combustion of smoking material so that the smoke may be tasted or inhaled for enjoyment or therapeutic purposes. Pipes include a bowl, a stem, and a mouthpiece. The bowl can be made of briar, corn, meerschaum, clay, glass, or other heat resistant materials.

Some pipes enable passing the smoke through a water basin, which filters out some materials in the smoke. A purge valve can be provided in the basin airspace.

### SUMMARY OF THE DISCLOSURE

In an embodiment of the disclosure, a pipe for smoking comprises a combustion bowl; a mouthpiece; an inhalation conduit; an inhalation check valve connected to the inhalation conduit and positioned between the bowl and the mouthpiece and oriented to pass smoke of combustion in one direction from the bowl to the mouthpiece; and an exhalation check valve positioned between the mouthpiece and the atmosphere and oriented to pass exhalation in one direction from the mouthpiece to the atmosphere.

In another embodiment of the disclosure, a pipe for smoking comprises a combustion bowl; a mouthpiece; an inhalation conduit; an inhalation check valve connected to the inhalation conduit and positioned between the bowl and the mouthpiece and oriented to pass smoke of combustion in one direction from the bowl to the mouthpiece; an exhalation check valve positioned between the mouthpiece and the atmosphere and oriented to pass exhalation in one direction from the mouthpiece to the atmosphere; and a body having an upper inner area and a lower inner area, the body being partially tillable to retain water in the lower inner area, the inhalation conduit having a first portion that passes from the bowl into the body and extending to the upper inner area and turning to extend to the lower inner area to form an open end that can be submerged by water that has filled the lower inner area, the inhalation conduit having a second portion having an opening in the upper inner area and extending to the inhalation check valve.

In a further embodiment of the disclosure, a method of forming a smoking pipe comprises connecting a combustion bowl to an inhalation conduit; connecting an inhalation check valve to the inhalation conduit, the inhalation check valve positioned between the bowl and the mouthpiece, the check valve oriented to pass smoke of combustion in one direction from the bowl to the mouthpiece; and connecting an exhalation check valve between the mouthpiece and a filter to pass exhalant in one direction from the mouthpiece to a filter.

### BRIEF DESCRIPTION OF THE DRAWINGS

A more complete understanding of the present disclosure, and the attendant advantages and features thereof, will be more readily understood by reference to the following detailed description when considered in conjunction with the accompanying drawings wherein:

FIG. 1 is a perspective view of a pipe of the disclosure;

FIG. 2 is a side view of the pipe of FIG. 1;

FIG. 3 is a perspective view of an alternative pipe of the disclosure;

FIG. 4 is a front view of the pipe of FIG. 1;

FIG. 5 is an alternative bowl configuration of the disclosure;

FIG. 6 is a bisecting cross section of the bowl of FIG. 5;

FIG. 7 is a cross-section of another alternative bowl configuration of the disclosure;

FIG. 8 is a perspective view of a bowl and cover of the disclosure;

FIG. 8A is an alternative cover and lighter of the disclosure;

FIG. 9 is a front view of an exhaust filter of the disclosure;

FIG. 10 is an exploded view of the exhaust filter of FIG. 9;

FIG. 11 is a top view of a mouthpiece of the pipe of FIG. 1, taken along line A-A of FIG. 4;

FIG. 12 is a cross section of a valve assembly of FIG. 11, depicting no air flow;

FIG. 13 is a cross section of the valve assembly of FIG. 12, during inhalation;

FIG. 14 is a cross section of the valve assembly of FIG. 12, during exhalation;

FIG. 15 depicts a duck-bill valve in the valve assembly of FIG. 12;

FIG. 16 depicts an alternative valve useable in the valve assembly of FIG. 12;

FIG. 17 is a cross section of an alternative mouthpiece useable in the pipe of FIG. 1, taken along line A-A of FIG. 4;

FIG. 18 is a side view of the mouthpiece of FIG. 17;

FIG. 19 is a side view of the manual valve of FIG. 17; and

FIG. 20 is a perspective view of a water pipe of the disclosure, the interior of the body shown in break-away.

### DETAILED DESCRIPTION OF THE DISCLOSURE

As required, detailed embodiments are disclosed herein; however, it is to be understood that the disclosed embodiments are merely examples and that the systems and methods described below can be embodied in various forms. Therefore, specific structural and functional details disclosed herein are not to be interpreted as limiting, but merely as a basis for the claims and as a representative basis for teaching one skilled in the art to variously employ the present subject matter in virtually any appropriately detailed structure and function. Further, the terms and phrases used herein are not intended to be limiting, but rather, to provide an understandable description of the concepts.

The terms “a” or “an”, as used herein, are defined as one or more than one. The term plurality. as used herein, is defined as two or more than two. The term another, as used herein, is defined as at least a second or more. The terms “including” and “having,” as used herein, are defined as comprising (i.e., open language). The term “coupled,” as used herein, is defined as “connected,” although not necessarily directly, and not necessarily mechanically.

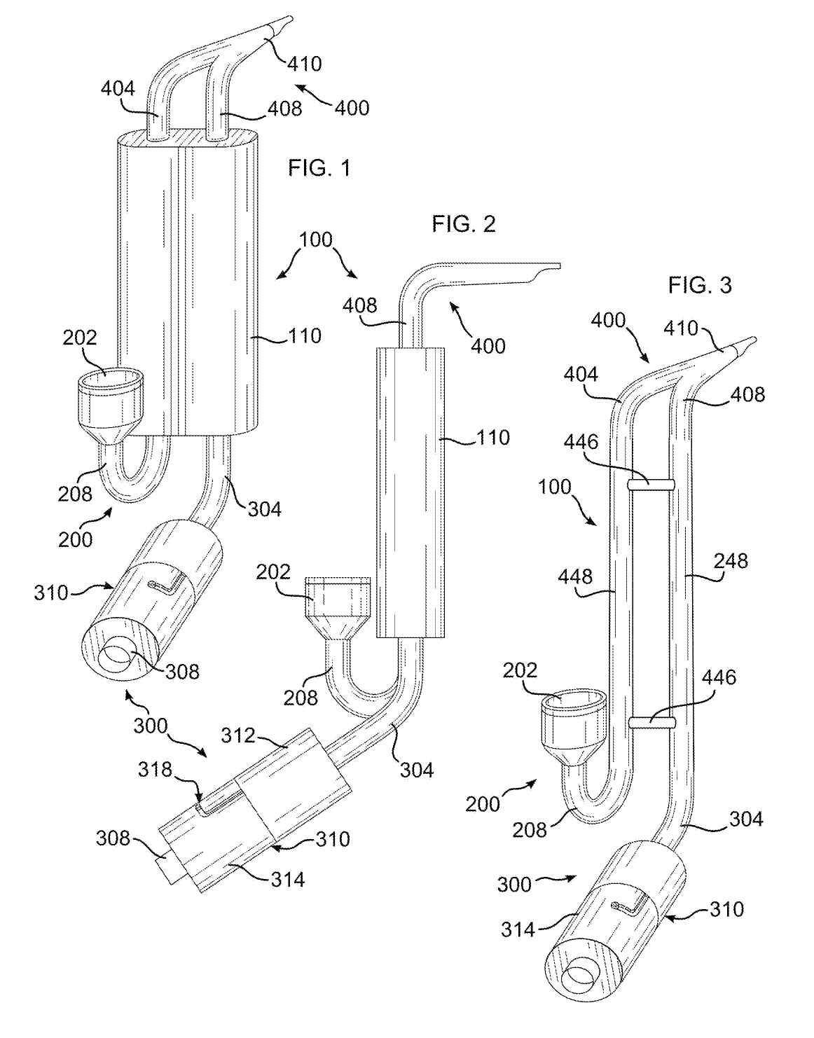

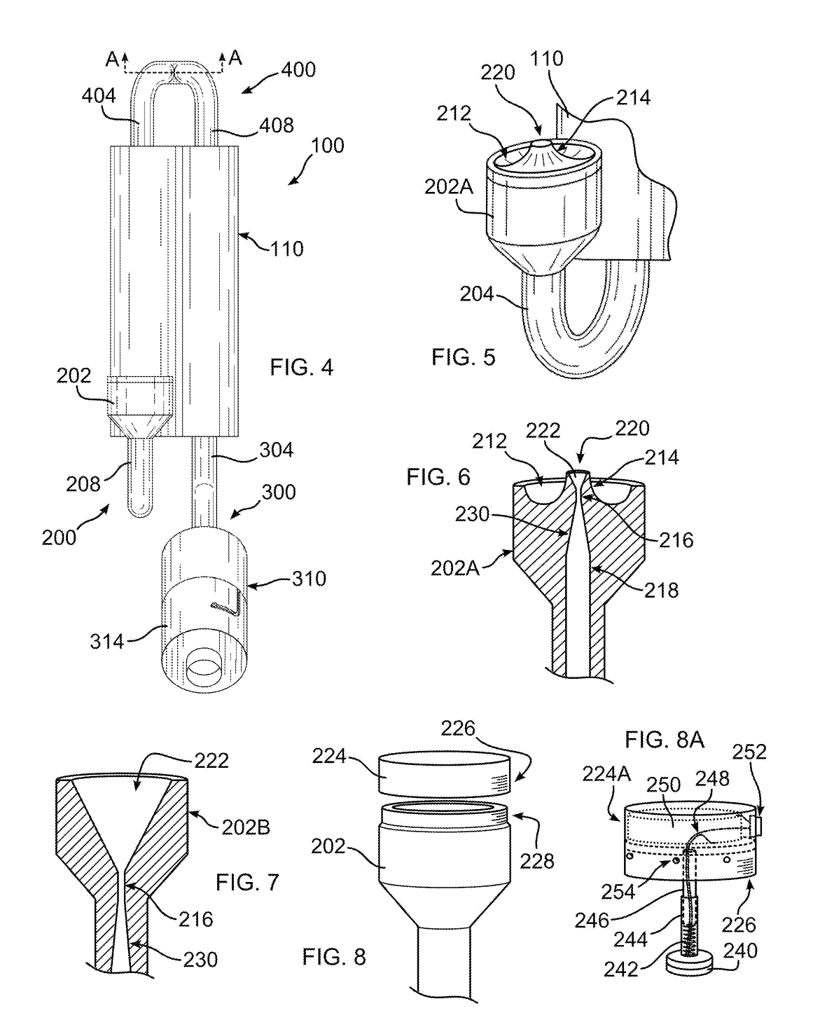

With reference to FIGS. 1-4, a smoking pipe 100 of the disclosure includes a main body 110 connected to a combustion assembly 200, an exhaust assembly 300, and a mouthpiece assembly 400. Pipe 100 can be used for smoking any type of smoking material, and enables filtering of smoke either before inhalation, after exhalation, or both.

Combustion assembly 200 includes a combustion chamber or bowl 202 connected to a combustion outlet conduit 208. In one embodiment, outlet conduit 208 extends directly to mouthpiece inlet conduit 404, as shown in FIG. 3. In another embodiment, combustion outlet conduit 208 discharges within an interior of body 110. Combustion outlet conduit 208 and mouthpiece inlet conduit 404 collectively form an inhalation conduit, and may be formed in one or more portions. Bowl 202 can include a slip fit conduit 206 (shown in FIG. 20) which nests within or around combutions outlet conduit 208, so that bowl 202 can conveniently be removed during smoking, for cleaning, or for replacement with a like or similar style bowl. Alternatively, slip fit conduit 206 can be positioned at any point along combustion outlet conduit 208, so that some or a portion of the combustion outlet conduit 208 can be grasped for removal and holding, rather than the relatively warmer bowl 202. In the illustrations, conduits form tubular cross sections, however they can have any cross sectional shape, and can be formed within blocks or other structures.

The shape of the interior or exterior of bowl 202 can be of any known shape or 100 configuration, or any known material. With reference to FIGS. 5-6, in one embodiment of the disclosure, a bowl 202A has an improved shape including a peripheral combustion ring 212 surrounding a raised nozzle 214 housing a combustion chamber 222. A narrow transfer channel 216 connects a bowl channel 218 to a nozzle outlet 220, which is conically shaped. When the pipe is drawn (during inhalation), a local vaccum is created to thereby draw smoke from burning 105 material within ring 212. Transfer channel 216 can be sized to reduce entry of ash or other debris, and can have a diameter as small as 0.01 inch, for example. Bowl channel 218 is sufficiently large such that a needle or other object can be used to push material through transfer channel 216 where it can easily fall through bowl channel 218 and be removed.

With reference to FIG. 7, in an alternative bowl shape of the disclosure, bowl 202B 110 includes a combustion chamber 222 which is cone shaped, tapering to transfer channel 216, the latter having the same properties as described for the embodiment of FIGS. 5-6. Transfer channel 216 can remain narrow until emptying into combustion outlet conduit 208, a bowl channel 218 can be provided.

FIGS. 6 and 7 illustrate a further embodiment of the disclosure, in which a Venturi effect is 115 created by providing an additional outlet taper 230 which follows or replaces the transfer channel 216. More specifically, the taper of combustion chamber 222 and the taper of outlet taper 230 can be chosen to cooperatively maximize a Venturi effect of the bowl 202A/202B. A Venturi effect is created because of the reduced diameter zone formed between the closing taper of combustion chamber 22 and the opening taper of outlet taper 230. This reduced diameter forms 120 the Venturi “choke”, and can be elongated by transfer channel 216 as shown, or transfer channel 216 can be shorter or eliminated. Advantageously, a taper of combustion chamber 222 is about 30 degrees, and the taper of outlet taper 230 is about 5 degrees, but these can vary considerably or even be eliminated, provided the choke formed by a narrower transfer channel 216 is present.

The Venturi effect causes smoke entering bowl channel 218 to increase speed as it passes through the reduced diameter zone, reducing the pressure within this zone. This contributes to drawing more air into combustion chamber 222, and greatly improves mixing of air with the smoke, resulting in an aerated smoke that can be more comfortable to inhale. FIGS. 6-7 illustrate that the overall dimensions of combustion chamber 222, transfer channel 216, and outlet taper 230 can vary considerably while providing the benefits described.

Combustion chamber 222 can be formed to be small, for example sized with a maximum diameter of less than a cm, for example 2 to 6 mm, to contain only sufficient material as it is desired to smoke at one occasion. For example, a small combustion chamber is advantageous for consuming a single dose of medicine to be smoked using pipe 100. Further, by forming combustion chamber to have a continuous taper, the desired quantity of smoking material to be consumed is concentrated together, to promote thorough burning. In this manner, medicine or other smoking material is not wasted and costs are reduced. The combination of a small combustion chamber and the aforedescribed Venturi effect help to ensure that the entire amount of the desired quantity is combusted and inhaled for a maximum therapeutic benefit or enjoyment.

FIG. 8 depicts a cap 224 which can be placed over any of the bowls 202 of the disclosure, and can attach with a slip fit, a snap fit, or mating threads 226, 228 on cap 224 and bowl 202, respectively. Cap 224 can be used for enclosing smoking material during transport or storage of pipe 100, extinguishing burning smoking material, or neatness of appearance and display.

In FIG. 8A, cap 224A is configured as a lighter, which can be inserted into smoking material to ignite it. An electric heating element 240 is sized to enter combustion chamber 222 and to press against the smoking material. A suitable safety cover, such as a retractable cover, not shown, can be provided to reduce a likelihood of heating element 242 touching a surface other than the smoking material. A biasing element 242, such as a coil spring, enables a predetermined force to be applied between the heating element and the smoking material as cap 224A is lowered towards combustion chamber 222. In the embodiment shown, telescoping poles 244, 246 provide further stability, and protection for wires 226, but are not required in all embodiments.

A battery 250 attached to cap 224A provides power for a heating element 240. Alternatively, a wire can extend from cap 224A to a power supply (not shown). Battery 250 can be recharged by a plug-in power supply, or can be recharged by inductive charging, where cap 224A includes a coil and associated electronics (not shown). An electrical switch 252, such as a momentary switch, closes an electrical circuit between heating element 240 and battery 250, and may be activated by the user when heating element is pressed against smoking material, and it is desired to cause heating element to be heated.

In an embodiment, one or more holes 254 are provided through cap 224A, whereby cap 224A can be attached to bowl 202, for example using a snap fit or threads 226, and fresh air for combustion can be drawn through holes 254. This reduces the release of unfiltered smoke to the environment. A single, larger hole can alternatively be provided, and this can act as a carburator or manual choke, as shown and described elsewhere herein with respect to air inlet valve 120 of FIG. 20.

In a further alternative embodiment, element 250 is a source of ignitable gas, telescoping legs 244, 246 operate a gas release valve (not shown), and heating element 240 may be a heating coil as shown, or can be an electrical spark igniter or a mechanical flint, whereby when cap 224A is pressed into smoking material, legs 244, 246 are compressed to release gas, and element 240 releases a spark or sufficient heat to ignite the gas, to thereby ignite the smoking material.

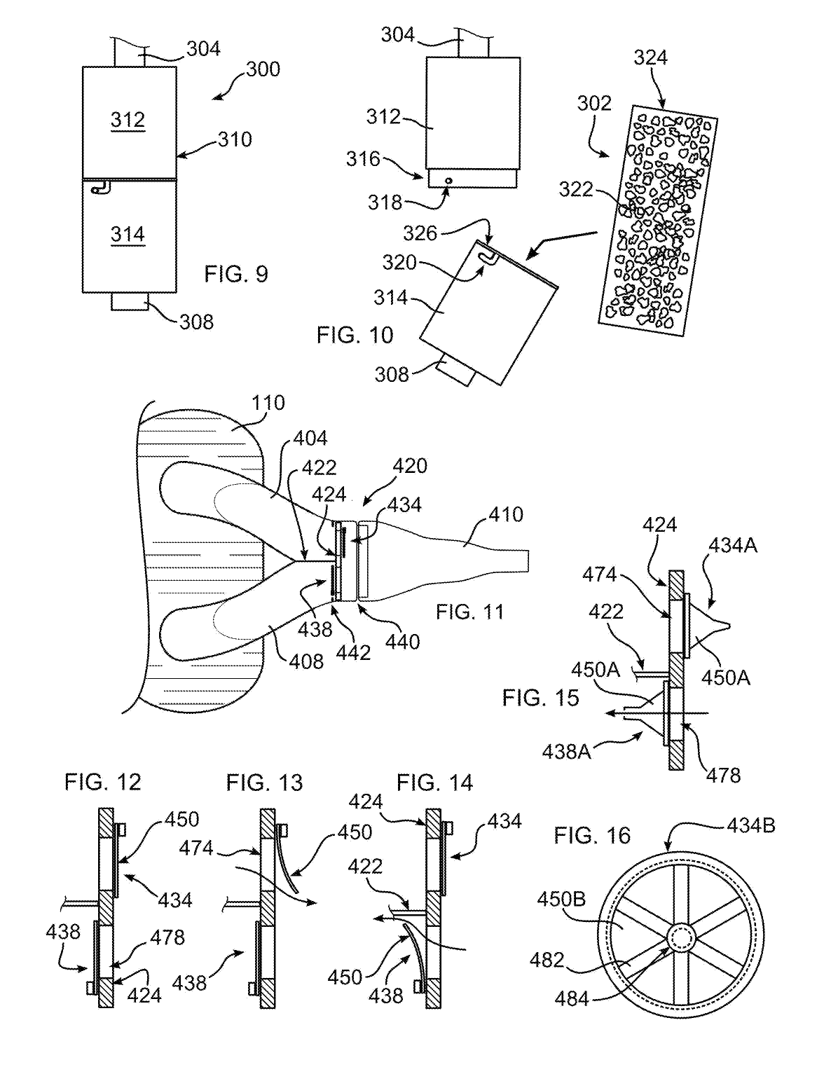

With reference to FIGS. 9-10, exhaust assembly 300 includes an exhaust chamber or canister 310 of any shape, in this embodiment being cylindrical, which is configured to be openable for the insertion of a filtering medium 302. An exhaust inlet conduit 304 conveys exhaled air together with unconsumed materials from mouthpiece outlet conduit 408 to an interior of canister 310, to be admixed with filtering medium 302. Canister 310 can be opened by any known means, including for example forming an end into a lid, or separating canister 310 into two mating sections 312, 314, as illustrated. An interference or slip fit 316 can be formed between the mating sections 312, 314, and the sections can further be maintained together by any known means, including for example threads, or a pin 318 which slides within an L-shaped groove 320, as illustrated. Exhaust inlet conduit 304 and mouthpiece outlet conduit 408 collectively form an exhalation conduit which can be formed in one or more portions.

Filtering medium 302 can be any material having the properties of removing unwanted elements from the exhaled matter, including for example smoke and its particulates, and odors. In an embodiment, filtering medium 302 includes activated charcoal or carbon 322 suspended within a matrix 324. The material for the matrix can advantageously be resilient in order to completely fill a diameter of canister 310 so that exhaust air cannot bypass carbon particles as it travels through the canister from inlet conduit 304 to exhaust outlet conduit 308. Filtering pouches or packages can be used to facilitate easy replacement of filter medium 302, or loose materials can be used. In an embodiment, a gasket 326 is provided to ensure a gas-tight seal of an opening in canister 310. Exhaust canister can be substantially larger or smaller than illustrated, depending on the extent of filtering desired for the exhalation material, and the efficiency of the filtering medium 302.

In an embodiment, exhaust assembly is separable from body 110 for cleaning or storage, for example at a slip fit 330 (shown in FIG. 20), or other form of attachment, including a threaded attachment. Where not needed, exhaust assembly 400 can be omitted, and exhaust inlet conduit 304 can form a stub forming an exhaust outlet, or can be omitted. In a further embodiment, an exhaust collecting bag can be attached to exhaust outlet conduit 308, or may be attached in lieu of exhaust assembly 300. The bag can be attached by any known means, including an elastic band, clamp, or clip. The bag can be composed of any material which will retain exhaust gases, or it can alternatively be composed of a filtering medium, such as a microporous medium, and can filter particulates and then release cleansed exhaust gases through its surfaces. The exhaust collecting bag can be any size, the size selected based upon whether it will filter gases, enabling a smaller bag, or whether it will collect gases, in which case it may be quite large, or resiliently expandable, or expandable by unfolding. The bag can include a reduced taper neck, in the manner of a party balloon, so that it can more easily form a tight seal around outlet conduit 308 or exhaust inlet conduit 304.

With reference to FIGS. 11-14, mouthpiece assembly 400 includes a mouthpiece inlet conduit 404, a mouthpiece 410, and a mouthpiece outlet conduit 408. Mouthpiece 410 can have any shape, however it should be convenient for a user of pipe 100 to both inhale and exhale 210 through mouthpiece 410. Such shapes can include those in which the lips are inserted into the mouthpiece, and those in which the lips surround the mouthpiece, an example of which is illustrated in the figures. In accordance with the disclosure, a valve assembly 420 is positioned between mouthpiece 410 and each of inlet and outlet conduits 404, 408. Valve assembly 420 enables a user to inhale substantially exclusively through inlet conduit 404, and to exhale substantially exclusively through outlet conduit 408. Thus, inhalation valve 434 and exhalation valve 438 each form a check valve. While inhalation valve 434 and exhalation valve 438 are shown side by side, inhalation valve 434 can be placed anywhere along a gas path between bowl 202 and mouthpiece 410, although this can cause a portion of the exhalant to be loaded into the inhalant path, which can be undesireable. Similarly, exhalation valve can be located distal from mouthpiece 410, although this could cause some waste of the inhalant. For this reason, valves 434, 438 are positioned close to mouthpiece 410, although this is not required.

Valve assembly 420, in one embodiment, includes a divider 422 which separates gas flow between inlet and outlet conduits 404, 408 and terminates at a valve plate 424 which separates gas flow from mouthpiece 410 to either conduit 404, 408 except through either an inhalation valve 434, or an exhalation valve 438. Divider 422 can be omitted if conduit 404 is directly connected to an input of valve 434, and conduit 408 is directly connected to an output of valve 438.

Valve plate 424 and valves 434, 438 can be inserted and removed as a unit. Mouthpiece 410 can be attached to mouthpiece assembly 400 by a slip fit connection 440, so that valve plate 424 can be inserted or removed, for cleaning, repair, or replacement. A projection 442 can be provided on an interior of mouthpiece assembly 400 to properly seat and orient valve plate 424, which can be held in place by friction or other means.

Portions of valve assembly 420 are shown in greater detail in FIGS. 12-14. In FIG. 12, neither valve 434, 438 are open, and there is no air flow through either valve. In FIG. 13, the user is inhaling, and air and combustion products flow in from mouthpiece inlet conduit 404, through a fixed inlet port 474, through valve 434, to the user. In this embodiment, valve 434 includes a resilient valve seal 450, which is anchored at one or more points, and can bend to admit air passage when exposed to air pressure.

There are various other forms of valve seals which are known or which may be hereafter discovered, and which can be used with the disclosure in place of valve seal 450. For example, valves 434A, 438A, shown in FIG. 15, include a duck bill valve seal 450A which forms a cone terminating in a linear opening which is normally resiliently held closed, but is expandable and deformable to admit passage of air in one direction only. Alternatively, example valve 434B in FIG. 16 illustrates a circular valve form 450B which includes a series of backing ribs 482 and a central anchor point 484 for the valve seal 450B.

In FIG. 14, the user is exhaling, whereby the pressure of exhalation forces open, exhalation valve 438, whereupon the exhaled air passes through fixed port 478 of valve plate 424, and through valve 438, to mouthpiece outlet conduit 408, and then through exhaust assembly 300 as described elsewhere herein, and to the atmosphere.

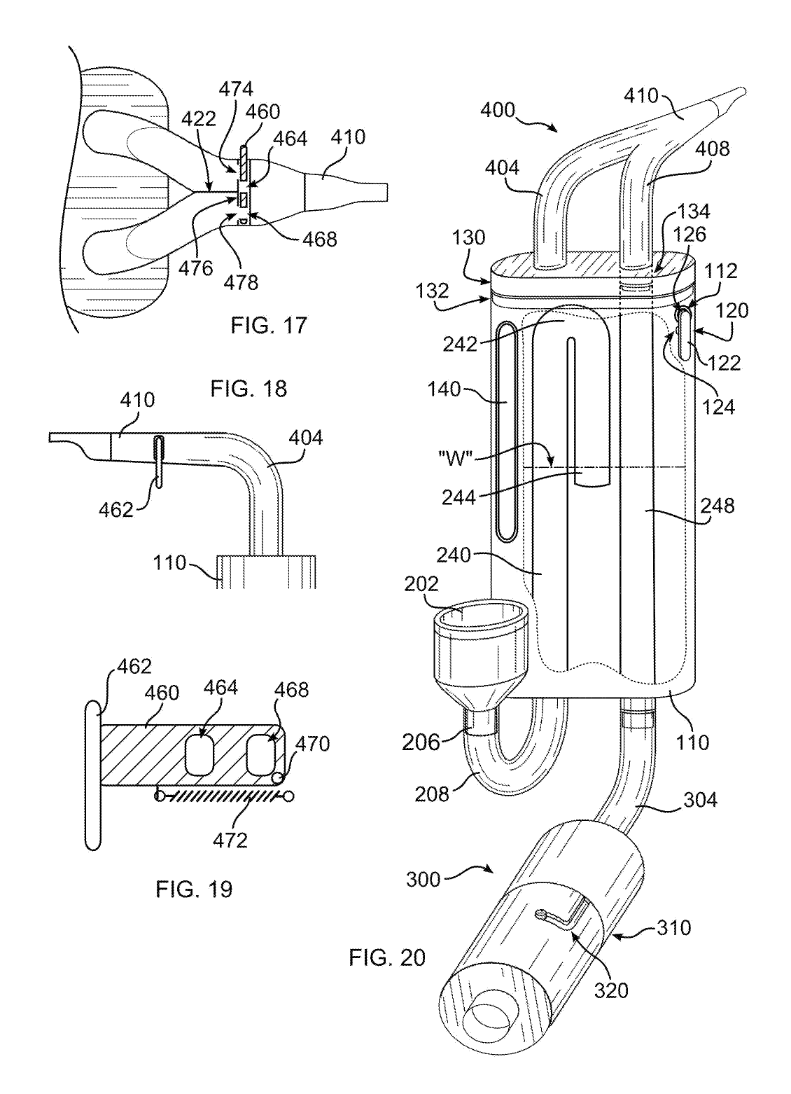

With reference to FIGS. 17-19, although valve assembly 420 is fully automatic and requires no user intervention, a manual valve can be provided which enables the user to determine when, and at what pressure, either the inlet valve 434 is open, or the exhaust valve 438 is open. More particularly, a sliding valve plate 460 includes a handle 462 which may be operated by the user to slide the valve plate 460 laterally to align either an inlet port 464 or an outlet port 468 with inlet conduit 404 or outlet conduit 408, respectively. A fixed port plate 476 has an fixed inlet port 474 and a fixed outlet port 478. When port 464 is not aligned with fixed inlet port 474, or port 468 is not aligned with fixed outlet port 478, air cannot flow past fixed port plate 476 to the respective conduit 404, 408. A valve plate pin 470 can be provided to prevent sliding valve plate 460 from being inadvertently removed. Multiple inlet ports and outlet ports can be aligned with multiple fixed inlet and outlet ports to define a shorter stroke, and a single port can serve for both an inlet port or an outlet port, however the stroke of sliding valve plate 460 would need to be longer to align this single port between a fixed inlet and outlet port.

A biasing member or spring 472, such as a wound wire or elastic band, can be provided to bias valve plate in either the inhalation or exhalation orientation, for example to protect valve plate 460 within an interior of mouthpiece 400 when not in use. Spring 472 can be connected at one end to valve plate 460, and at another end to an interior of mouthpiece 400.

Referring now to FIG. 20, pipe 100 has the form of a water pipe, body 110, shown in partial cutaway, being fillable with a quantity of water which can be used to filter and change a temperature of the smoke to be inhaled. Components can be as described elsewhere herein, unless as modified as follows. In particular, combustion outlet conduit 208 enters into body 110 and forms or is joined to a dispersion conduit 240 which passes through liquid contained within body 110 and then reverses direction at bend 242, to discharge at a dispersion conduit outlet 244. Water is added to body 110 to be at a level not far above dispersion conduit outlet 244, as shown by water line indicating line “W”. Mouthpiece inlet conduit 404 can have a portion projecting 275 into body 110, however the length should be substantially above line “W”. As such, an upper area of body 110 is defined to include an area which is not intended to be under water, and a lower area of body 110 is defined to include an area at least part of which is intended to be under water, when body 110 is filled with water to submerge dispersion conduit outlet 244.

Mouthpiece outlet conduit 408 can be connected to canister inlet conduit 304 (or an exhaust port) by passing directly through body 110 through an exhaust connecting conduit 248. Alternatively, mouthpiece outlet conduit 408 can be connected to canister inlet conduit 304 completely outside body 110.

Body 110 is sealed except for the admission of smoke via combustion outlet conduit 208, or dispersion conduit outlet 244. For smoking with water, it is typically desired to charge a chamber with fresh smoke, and to then admit fresh air into the chamber as the accumulated smoke is inhaled. For this purpose, bowl 202 can be removed to admit fresh air through combustion outlet conduit 208. Alternatively, a carb or air inlet 112 can be provided, having the form of a hole that is simply covered with a finger, or a valve as illustrated. More particularly, air inlet valve 120 includes a rocker arm 122 which pivots on a base 124, and which includes a stopper 126 sized to seal inlet 112. A spring (not shown) biases rocker arm 122 to seal inlet 112. Valve 120 can be a one-way valve which is closed when the pipe is drawn during inhalation through mouthpiece 410, and which opens when purging stale smoke by gently blowing into pipe through mouthpiece 410. A one-way valve of any of the types shown in FIGS. 11-16 can be used for this purpose, or another type of one-way valve.

A cover 130 engages a remainder of body 110 to form a gas and watertight seal. In one embodiment, a resilient slip fit 132 is formed, however a seal can be formed by other methods. Mouthpiece inlet and outlet conduits 404, 408 can be connected to cover 130, and outlet conduit 408 can be separable from exhaust connecting conduit 248 at a slip fit connection 134, or by other means, at an interior or under side of cover 130. An optional observation window 140 is positioned vertically along body 110, enabling a user to observe the water level and appearance, as well as the quantity and density of the smoke charge within body 110. In an embodiment, body 110 is transparent to facilitate these functions.

To enable setting pipe 100 down in a vertical position when containing water, it is possible to configure combustion outlet conduit 208 and, if present, exhaust inlet conduit 304, to exit from a side surface of body 110, leaving a flat lower surface. Alternatively, a suitable holder can be provided which supports pipe 100 in a sufficiently vertical position upon a surface.

Body 110 and conduits 208, 404, 408, and 304 can be fabricated from plastic, wood, metal, glass, ceramic, or any other material of sufficient strength and water and chemical resistance. In an embodiment, the foregoing elements are fabricated from stainless steel or brass. Connections between components can be carried out by soldering, brazing, welding, crimping, adhesive, fastener, or any other means.

A flexible hose can be connected in place of mouthpiece 410, and another mouthpiece 410 can be provided at the end of the hose. A plurality of such hoses can be provided, connected to a single body 110.

FIG. 3 illustrates an embodiment of pipe 100 which does not include a body, and in which conduits 208 and 404 are directly connected by a combustion connecting conduit 448, and conduits 408 and 304 are directly connected by exhaust connecting conduit 248. Connector bars 446 join connecting conduits 248 and 448 to form a unitary structure.

Thus, pipe 100 of the disclosure enables filtering of a smoke charge in water or other liquid, and filtering of the exhalation by a filter. This enables a more enjoyable smoking experience, and a reduced impact to the environment or to other people who do not wish to be exposed to the odor, chemicals, or smoke associated with smoking.

All references cited herein are expressly incorporated by reference in their entirety. It will be appreciated by persons skilled in the art that the present disclosure is not limited to what has been particularly shown and described herein above. In addition, unless mention was made above to the contrary, it should be noted that all of the accompanying drawings are not to scale. There are many different features to the present disclosure and it is contemplated that these features may be used together or separately. Thus, the disclosure should not be limited to any particular combination of features or to a particular application of the disclosure. Further, it should be understood that variations and modifications within the spirit and scope of the disclosure might occur to those skilled in the art to which the disclosure pertains. Accordingly, all expedient modifications readily attainable by one versed in the art from the disclosure set forth herein that are within the scope and spirit of the present disclosure are to be included as further embodiments of the present disclosure.

What is claimed is:

1. A pipe for smoking, comprising:
a combustion bowl;
a mouthpiece;
an inhalation conduit;
an inhalation check valve connected to the inhalation conduit and positioned between the bowl and the mouthpiece and oriented to pass smoke of combustion in one direction from the bowl to the mouthpiece; and
an exhalation check valve positioned between the mouthpiece and the atmosphere and oriented to pass exhalation in one direction from the mouthpiece to the atmosphere.

2. The pipe of claim 1, further including an exhaust filter connected to the exhalation check valve and having a chamber containing a filter material interposed between the exhalation check valve and an exhaust.

3. The pipe of claim 2, wherein the filter material includes activated carbon.

4. The pipe of claim 2, wherein the chamber has an opening through which filter material may be inserted into the chamber.

5. The pipe of claim 1, wherein the exhaust filter is connected to the exhalation valve by an elongate conduit.

6. The pipe of claim 1, wherein the mouthpiece includes a chamber connected to the inhalation check valve and the exhalation check valve on a first end, and passage leading outside of the mouthpiece on a second end.

7. The pipe of claim 1, wherein at least one of the inhalation check valve and the exhalation check valve include a flexible membrane that is bent to admit passage of inhalant or exhalant, respectively.

8. The pipe of claim 1, wherein at least one of the inhalation check valve and the exhalation check valve include a duck-bill valve.

9. The pipe of claim 1, wherein the bowl includes a peripheral combustion chamber having a base and an upper extent, the peripheral combustion chamber surrounding a central column having a channel with an opening positioned at a level higher than the upper extent.

10. The pipe of claim 1, further including a cap threadable onto the bowl.

11. The pipe of claim 1, further including an exhalation conduit connected to the exhalation check valve at a first end, wherein the inhalation conduit and the exhalation conduit are formed as separate and distinct conduits.

12. The pipe of claim 11, wherein the inhalation conduit and the exhalation conduit are elongated and are mutually attached.

13. The pipe of claim 11, wherein the inhalation conduit and exhalation conduit are connected to a body forming an inner chamber.

14. The pipe of claim 13, wherein the body has an upper inner area and a lower inner area, the body being partially fillable to retain water in the lower area, the inhalation conduit having a first portion that passes from the bowl into the body and extending to the upper inner area and turning to extend to the lower inner area to form an open end that can be submerged by water that has filled the lower inner area, the inhalation conduit having a second portion having an opening in the upper inner area and extending to the inhalation check valve.

15. The pipe of claim 14, further including a port formed in the body to enable passage of air from the atmosphere into the upper area.

16. The pipe of claim 15, wherein the port is closeable by a rocker arm and stopper.

17. The pipe of claim 14, the body further including an elongate transparent window extending between the lower area and the upper area.

18. A pipe for smoking, comprising:
a combustion bowl;
a mouthpiece;
an inhalation conduit;
an inhalation check valve connected to the inhalation conduit and positioned between the bowl and the mouthpiece and oriented to pass smoke of combustion in one direction from the bowl to the mouthpiece;
an exhalation check valve positioned between the mouthpiece and the atmosphere and oriented to pass exhalation in one direction from the mouthpiece to the atmosphere; and
a body having an upper inner area and a lower inner area, the body being partially fillable to retain water in the lower inner area, the inhalation conduit having a first portion that passes from the bowl into the body and extending to the upper inner area and turning to extend to the lower inner area to form an open end that can be submerged by water that has filled the lower inner area, the inhalation conduit having a second portion having an opening in the upper inner area and extending to the inhalation check valve.

19. The pipe of claim 19, further including an exhalation conduit positioned between the exhalation check valve and a filter.

20. A method of forming a smoking pipe, comprising:
connecting a combustion bowl to an inhalation conduit;
connecting an inhalation check valve to the inhalation conduit, the inhalation check valve positioned between the bowl and the mouthpiece, the check valve oriented to pass smoke of combustion in one direction from the bowl to the mouthpiece; and
connecting an exhalation check valve between the mouthpiece and a filter to pass exhalant in one direction from the mouthpiece to a filter.
